(19) United States
(12) Patent Application Publication
(10) Pub. No.: US 2006/0234336 A1
(43) Pub. Date: Oct. 19, 2006

(54) Methylotrophic bacterium for the production of recombinant proteins and other products

(76) Inventors: Carlos B. Miguez, Pierrefonds (CA); Marianne M. Figueira, Laval (CA); Louise Laramee, Lasalle (CA); Colin J. Murrell, Coventry (CA)

Correspondence Address: OGILVY RENAULT LLP, 1981 MCGILL COLLEGE AVENUE, SUITE 1600, MONTREAL, QC, H3A2Y3, CA

(21) Appl. No.: 10/497,060

(22) PCT Filed: Nov. 29, 2002
(86) PCT No.: PCT/CA02/01840

§ 371 Date: Nov. 29, 2004

(30) Foreign Application Priority Data
Nov. 30, 2001 (CA) 2364043
Dec. 3, 2001 (US) 09998631
Jul. 5, 2002 (US) 10188746

(51) Int. Cl. C12P 21/06; C12N 1/21; C07H 21/04; C12N 9/00; C07K 14/195; C12N 15/74

(52) U.S. Cl. 435/69.1; 435/471; 435/183; 435/252.3; 530/350; 536/23.2

(57) ABSTRACT

The present invention relates to a method of producing a recombinant peptide, a recombinant protein, or a product from metabolic engineering using a genetically modified methylotrophic bacterium, and more particularly to Methylobacterium extorquens ATCC 55366. The method comprises introducing am expression vector into the methylotrophic bacterium, the expression vector comprising a polynucleotide sequence, coding for a peptide or protein, or allowing for production of a product from metabolic engineering under the control of a regulated promoter. the method also comprises growing the genectically modified methylotrophic bacterium in a minimal salts medium lacking organic sugars and containing methanol. A metal ion may be used for regulating the expression of the polynucleotide sequence by the promoter.

### CROSS REFERENCE TO RLATED APPLICATIONS

The present application claim benefit under 35 U.S.C. §120 of earlier application Ser. No. 10/188,746 filed Jul. 5. 2002 and of application Ser. No. 09/998,631 filed Dec. 3, 2001, both pending, the entire content of both of which is hereby incorporated by reference.

### TECHNICAL FIELD

This invention relates to a system and method of producing recombinant peptides or proteins and other products from metabolic engineering in prokaryotes. More specifically, the present invention relates to the use of the methylotrophic bacterium Methylobacterium extorquens ATCC 55366, in combination with novel expression vectors, as an efficient expression system for recombinant peptides or proteins and industrially important bulk chemicals.

### BACKGROUND OF THE INVENTION

Current microbial processes for the production of recombinant proteins use either eukaryofic microorganisms (e.g. Pichia pastoris), which may produce unwanted glycosylation and other unwanted post-translational modifications, or prokaryotic cells. The bacterium Escherichia coli is the best known and the most used prokaryotic expression system. The E. coli system, however, has drawbacks that include inclusion body formation (when undesired), high acetate production which tends to inhibit growth and product formation, and a requirement for relatively expensive carbon sources such as glucose.

Methylotrophic bacteria are a group of prokaryotic microorganisms that can utilize one-carbon (C₁) compounds more reduced than carbon dioxide as a source of carbon and energy. Formaldehyde, an intermediate in the oxidation of reduced C₁ compounds, is incorporated into cells carbon via the serine pathway or via other pathways, and/or can be further oxidized in a series of reactions to CO₂, generating energy in the form of reducing equivalents.

Methylobacterium extorquens ATCC 55366 is a pink-pigmented faculative methylotroph isolated from a hydrocarbon-contaminated sandy soil [Bourque et al (1992) Appl. Microbiol. Biotechnol. 37:7-12]. The growth of this bacterium in a fed-batch fermentation system developed by Bourque et al [Bourque et al (1995) Appl. Microbiol. Biotechnol. 44(34): 367-376] resulted in cultivation at very high cell densities using a relatively cheap substrate, methanol, for the production of poly-β-hydroxybutyrate, a very interesting polyester.

The ability to produce high biomass densities in fermenters, combined with the newly acquired genetic information obtained from the genome sequencing of M. extorquens AM1 [Alper (1999) Science 283:1625-1626], renders this microorganism extremely interesting as a potential expression system for recombinant peptides or proteins and for the production of industrially important bulk chemicals. In order to achieve these objectives, it is essential to identify efficient cloning vectors and promoters for introducing new genes into M. extorquens.

It would be highly desirable to be provided with an efficient cloning vectors and promoters for introducing new genes into M. extorquens.

It would also be highly desirable to be provided with a method for the production of a large variety of products from metabolic engineering, which would help overcome some of the current problems.

### SUMMARY OF THE INENTION

One aim of the present invention is to provide an efficient cloning vectors and promoters for introducing new genes into M. extorquens.

Another aim of the present invention is to provide a method for the production of products from metabolic engineering, which would help overcome some of the problems faced with current microbial processes.

Such methods would include a new non-pathogenic prokaryotic microbial system, as an alternative to E. coli, for recombinant peptide or protein expression, which utilizes methanol as a source of carbon and energy for growth in chemically, protein-free, defined medium. Such methods could use a minimal culture medium combined with methanol as a carbon and energy source, which would lower the costs of producing products from metabolic engineering. Such systems would also include a new prokaryotic microbial system capable of producing recombinant peptides or proteins and other products from metabolic engineering at levels comparable to P. pastoris (grams/liter) in a high cell density fermentation process from methanol. Such methods would further include the development of transformation vectors, which would be stably or satisfactorily maintained in the cells in the presence of selective pressure such as antibiotics.

In accordance with one preferred embodiment of the present invention there is provided a method of producing a recombinant peptide, a recombinant protein or a product from metabolic engineering using a genetically modified methylotrophic bacterium under the control of a regulated promoter from a methylotrophic microorganism of the same or different species as the methylotrophic bacterium, said method comprising the steps of:

a) introducing into said methylotrophic bacterium an expression vector comprising a polynucleotide sequence coding for a peptide or a protein under the control of a regulated promoter for producing said peptide or protein or for allowing production of a product from metabolic engineering; and

b) growing said genetically modified methylotrophic bacterium in a minimal salts medium lacking organic sugars and containing methanol as a source of carbon and energy for said bacterium for a time sufficient to allow production of said peptide or protein or said product from metabolic engineering.

In one embodiment, the method of the present invention further comprises the step of:

c) regulating expression of the polynucleotide sequence by the promoter.

In another embodiment of the present invention the regulated promoter is a metal regulated promoter and step c) is effected with a metal ion.

In another embodiment of the present invention the methylotrophic bacterium is of the species Methylobacterium.

In another embodiment of the present invention the methylotrophic bacterium is Methylobacterium extorquens ATCC 55366.

In one embodiment of the present invention, the polynucleotide sequence is a gene coding for green fluorescent protein.

In another embodiment of the present invention, the polynucleotide sequence is a gene coding for an enzyme.

In another embodiment of the present invention, the polynucleotide sequence is a gene that encodes a peptide or protein that is not an enzyme.

In one embodiment of the present invention, the enzyme reads with a component within or from the culture medium to produce a peptide or protein, or other product from metabolic engineering.

In another embodiment of the present invention, the peptide or protein, or other product from metabolic engineering, reacts with a component within or from the culture medium to produce a product from metabolic engineering.

In one embodiment of the present invention, the polynucleotide sequence is inserted into a vector suitable for introduction into a methylotrophic bacterium, wherein the vector is stably maintained within the methylotrophic bacterium during growth and replication of the methylotrophic bacterium in the presence of selection pressure, and wherein the vector allows for the expression of the polynucleotide sequence within the methylotrophic bacterium.

In one embodiment of the present invention, the selective pressure is an antibiotic.

In one embodiment of the present invention, the regulating expression of the polynucleotide sequence by the promoter is with Cu.

In one embodiment of the present invention, the promoter is the promoter present in the soluble methane monooxygenase (sMMO) operon of Methylosinus trichosporium OB3b.

In another embodiment of the present invention, the promoter is pmxaF from a gene from a methylotrophic bacterium.

In a further embodiment of the present invention, the promoter is a promoter from a gene from a methylotrophic bacterium.

In another embodiment of the present invention, the promoter is a promoter from a gene from an organism other than a methylotrophic microorganism.

In one embodiment of the present invention, the expression vector is pmmoX-GFP-pRK310.

In another embodiment of the present invention, the expression vector is pmmoX-GFP-pVK101.

In another embodiment of the present invention, the expression vector is pLac-GFP-pJB3KmD.

In a further embodiment of the present invention, the expression vector is pmxaF-GFP-pCM110.

In another embodiment of the present invention, the expression vector is pLac-GFP-pRK310.

In one embodiment of the present invention, the present invention can be used for high-throughput peptide or protein production, or high-throughput production of other products from metabolic engineering.

In another embodiment of the present invention, the present invention can be used for proteomics-based peptide or protein expression or proteomics-based expression of other products from metabolic engineering.

In one embodiment of the present invention, the growing the genetically modified methylotrophic bacterium is performed within a flask.

In another embodiment of the present invention, the growing the genetically modified methylotrophic bacterium is performed within a fermenter.

For the purpose of the present invention the following terms are defined below.

The term “methylotrophic bacterium” is intended to mean a group of prokaryotic microorganisms that can utilize one-carbon (C₁) compounds more reduced than carbon dioxide as a source of carbon and energy.

The term “GFP” is intended to mean green fluorescent protein.

The term “expression vector” is intended to denote a DNA molecule, linear or circular, that comprises a segment encoding a polypeptide of interest operably linked to additional segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both.

The term “operably linked”, when referring to DNA segments, denotes that the segments are arranged so that they function in concert for their intended purposes, e.g. transcription initiates in the promoter and proceeds through the coding segment to the terminator.

The term “polynucleotide” denotes a single or double-stranded polymer of deoxyribonucleotide or ribonucleotide bases read from the 5′ to the 3′ end. Polynucleotides include RNA and DNA, and may be isolated from natural sources, synthesized in A, or prepared from a combination of natural and synthetic molecules. Sizes of polynucleotides are expressed as base pairs (abbreviated “bp”), nucleotides (“nt”), or kilobases (“kb”). Where the context allows, the latter two terns may describe polynucleotides that are single-stranded or double stranded. When the term is applied to double-stranded molecules it is used to denote overall length and will be understood to be equivalent to the term “base pairs”. will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

The term “polypeptide” is intended to denote a polymer of amino acid residues joined by peptide bonds, whether produced naturally or synthetically, Polypeptides of less than about 10 amino acid residues are commonly referred to as “peptides”.

The term “promoter” is intended to denote a portion of a gene containing DNA sequences that provide for the binding of RNA polymerase and initiation of transcription. Promoter sequences are commonly, but not always, found in the 5′ non-coding regions of genes.

The term “facultative methylotroph” is intended to denote a bacterium able but not obliged to grow in and perhaps use methanol as a carbon and/or energy source, but will also survive and perhaps grow in the absence of methanol.

The term “obligate methylotroph” is intended to denote a bacterium obliged to grow in and perhaps use methanol as a carbon and/r energy source, the bacterium will not survive or grow i the absence of methanol.

The term “and other products from metabolic engineering” is intended to mean, without limitation, plasmids for gene therapy or to support R&D activities, enzymes (cellulases, proteases, lipases), pigments (beta carotene, food colorants, antioxidants), vitamins (vitamin B12, biotin, riboflavin), amino acids (lysine, tryptophane, tyrosine, alanine), polysaccharides (pullulan, cellulose, chifin), biosurfactants (rhamnolipids, emulsan), biopestcides (Bt toxins, TMOF), hormones (insulin), antibiotics (tetracycline, penicillin, gramicidin, kanamycin). and biomaterials (silk, elastin, albumins).

### BRIEF DESCRIPTION OF RHE DRAWINGS

FIG. 1 illustrates the schematic strategy used to create different GFP-carrying plasmids.

FIG. 2 illustrates GFP production by M. extorquens in LB or CHOI media.

FIG. 3 illustrates GFP production by M. extorquens dependent on copper concentration in the medium. Bars represent the error deviation within four independent fluorescence measurements.

FIG. 4 illustrates the production of GFP during the growth of M. extorquens (clone 3-63, pmmoX-GFP-pVK101 construct). Bars represent the error deviation within four independent fluorescence measurements.

FIGS. 5A to 5C illustrate growth of Methylobacterium extorquens clone M123A in 9-L fed-batch fermentation in the presence (●) or absence (▴) of tetracycline, expressed in terms of biomass yield (g cell dry weight (CDW)/L) (FIG. 5A), GFP production (mg protein/liter) (FIG. 5B) and GFP specific yield (mg protein/g CDW) (FIG. 5C)

FIGS. 6A to 6C illustrate growth of Methylobacterium extorquens clone 23-16 in 9-L fed-batch fermentation in the presence (●) or absence (▴) of tetracycline, expressed in terms of biomass yield (g cell dry weight (CDWYL) (FIG. 6A), GFP production (g protein/liter) (FIG. 6B) and GFP specific yield (mg protein/g CDW) (FIG. 6C).

### DETAILED DESCRIPTION OF THE INVENTION

The present invention relates to the use of a new prokaryotic expression system that can overcome drawbacks inherent in using current eukaryotic or prokaryotic cells for the production of recombinant peptides or proteins and other products from metabolic engineering. In particular, the present invention relates to the use of various expression vectors that can be used for recombinant peptide or protein expression and production of other products from metabolic engineering in M. extorquens. M. extorquens is a prokaryotc methylotrophic bacterium known to lead to high biomass densities in fermenters and whose genome has been completely sequenced. This microorganism is, therefore, extremely attractive as a potential expression system. The present invention provides a new prokaryotic microbial system capable of producing recombinant peptides or proteins and other products from metabolic engineering at high levels in a high cell density fermentation process from methanol.

Bacterial Strains, Plasmids and Growth Conditions

M. extorquens ATCC 55366 [Bourque et al. (1992) Appl. Microbiol. Biotechnol. 37:7-12] was grown as described previously [Bourque et al. (1995) Appl. Microbiol. Biotechnol. 44(304):367-376]. The ATCC number 55366 is the number designated to the purified specimen culture deposited on Oct. 14, 1992 with the American Type Culture Collection (ATCC, 12301 Parklawn Drive, Rockville, Md. 20852, United States of America). Electro-competent cells of M. extorquens were prepared by the method of Toyama et al. [Toyama et al. (1998) FEMS Microbiol. Lett. 166(1):1-7] after slight modifications. Cells were grown in CHOI medium (containing 1% v/v methanol) until the culture reached an OD₆₀₀≈0.6-0.8. Cells were harvested by centrifugation (1800×g, 10 min, 4° C.) and washed twice with ice-cold sterile 10% (v/v) glycerol solution. The cell suspension was concentrated 10-fold in 10% glycerol. dispensed in 400-μl aliquots and kept at −80° C. Electro-competent cells (100 μl) were mixed with DNA solution (500 ng) in a 0.2-cm cuvette chilled on ice. Electroporation was carried out using a Gene Pulser (Bio-Rad) with the following parameters: 2.5 kV, 400Ω, 25 μF, to a final field strength of 12.5 kV cm⁻¹. After cells had been pulsed, 1 ml of ice-cold sterile Luria-Bertani low salts (LBLS) medium was immediately added to the cuvette, the cell suspension transferred into a test tube, and then incubated at 30° C. for 24 h. Transformed clones were selected in LBnS (Luria-Bertani without NaCl) agar medium with appropriate antibiotics (kanamycin, 50 μg ml⁻¹; tetracycline, 20 μg ml⁻¹). Escherichia coli DH5α (Life Technologies Gibco BRL) or Top 10 (Invitrogen) for pCM constructs was cultivated at 37° C. in LBLS broth or on agar plates. Plasmids in E. coli were selected with ampicillin (100 μg ml⁻¹), kanamycin (50 μg ml⁻¹) or tetracycline (20 μg ml⁻¹), Information on the plasmids used for the present invention is given in Table 1.

TABLE 1
Plasmids used

| Plasmid | Description | Source |
|---|---|---|
| pJB3KmD^{a} | cloning vector, IacZ′, oriV, onT, Ap^{┌}, | [1] |
| | Km, 6.1 kb | |
| PRK310 | cloning vector, IacZ′, oriV, onT, Tc^{┌}, | [2] |
| | 19 kb | |
| PVK101 | cloning vector, IacZ′, oriV, Tc^{┌}, Km^{┌}, | [3] |
| | 20 kb | |
| PMTL1000 | cloning vector, IacZ′, ori, Ap^{┌}, | [4] |
| | ˜12 kb | |
| PQBI63 | expression vector, 77, ori, Ap^{┌}, | [5] |
| | 6.3 kb | |
| PCM110 | cloning vector, PmxaF, oriV, onT, | [6] |
| | Tc^{┌}, ˜5.8 kb | |
| GFP-pMTL1000 | pMTL1000 with 0.78-kb insert | This |
| | containing GFP coding sequence | invention |
| | from pQBI63 | |
| PLac-GFP-pJB3KmD | pKJ3KmD with 0.78-kb insert | This |
| | containing GFP coding sequence | invention |
| | from pQBI63 | |
| PLac-GFP-pRK310 | pRK310 with ˜0.78-kb insert | This |
| | containing GFP coding sequence | invention |
| | from pQBI63 | |
| PmmoX-GFP- | pRK310 with ˜9.5-kb insert | This |
| pRK310 | containing sMMO and GFP coding | invention |
| | sequence from GFP-pMTL1000 | |
| PmmoX-GFP- | pVK101 with ˜9.5-kb insert | This |
| pVK101 | containing sMMO and GFP coding | invention |
| | sequence from GFP-pMTL1000 | |
| PmxaF-GFP-pCM110 | PCM110 with ˜1-kb insert | This |
| | containing GFP coding sequence | invention |
| | from pQBI63 | |

Ap^{┌}, Km^{┌}, Tc^{┌} denote resistance to ampicillin, kanamycin and tetracyclin, respectively.
^{a}Accession Databank No. U75323.
[1] Blatny et al. (1997) Appl. Environ. Microbiol. 63(2):370-379.
[2] Toyama et al. (1998) FEMS Microbiol. Lett. 166-(1):1-7.
[3] Knauf and Nester (1982) Plasmid 8:45-54.
[4] Nielsen et al. (1997) Mol. Microbiol. 25(2):399-409.
[5] Quantum Biotechnologies, Inc. (1998) Autofluorescent Proteins: Applications Manual. 11NO98.
[6] Marx et al., (2001) Microbiology 147, 2065-2075.

Construction of Plasmids

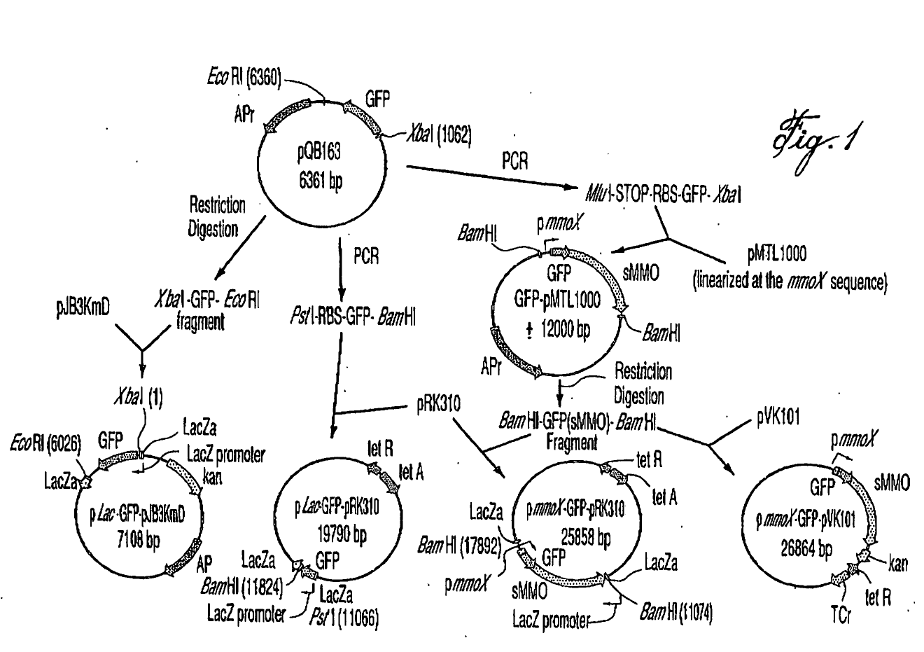

vitro DNA manipulation for cloning in E. coli was performed as described by Sambrook et al. [Sambrook et al. (1989) Molecular Cloning: A Laboratory Manual. Cold Spring Harbor Laboratory, Cold Spring Harbor, N.Y.]. The strategy used to create different GFP carrying plasmids (except PmxaF-GFP-pCM110, see further below) is represented in FIG. 1. The set of primers used were: (a) GFP/BamH1.2 (5′-GAA TCG GGA TCC TCA GTT GTA CAG TTC ATC CAT GG-3′; BamHI restriction site underlined; SEQ ID NO:1) and RBS/Psti.2 (5′-AAC AAA CTG CAG AAT AAT TTT GTT TAA CTT TAA GAA GG-3′; Pstl restriction site underlined; SEQ ID NO:2); and (b) RBS/Mlul (5′-CAC GAC GCG TTG AAA TAA TTI TGT TTA ACT TTA AGA AGG-3′, Mlul restriction site underlined; SEQ ID NO:3) and GFP/Xbal (5′-TGC TCT AGA TCA GTT GTA CAG TTC ATC CAT GC-3′, Xbal restriction site underlined; SEQ ID NO:4). The polymerase chain reaction conditions in both cases were: hot start at 94° C. for 2 min and then 30 cycles of amplification (94° C., 30 s; 55° C., 30 s; 72° C., 30 s) followed by a final extension at 72° C. for 10 min.

Detection of GFP Expression in M. extorquens

Selected clones of M. extorquens carrying GFP constructs were grown in LBnS or in CHOI medium containing 1% methanol and the appropriate antibiotic (kanamycin, 20 μg ml⁻¹; tetracycline, 20 μg ml⁻¹) at 30° C., 250 rpm. After 72 h of incubation, cells were harvested by centrifugation and washed twice with sterile, deionized water. Cells were resuspended in 700 μl of water and two 100 μl aliquots were dispensed into 96-well plates. The remaining 500 μl was used to determine cell dry weight. Cells harboring pJB3KmD, pRK310, pVK101 or pCM110 were used as control, and their fluorescence was subtracted from values obtained with cells harboring plasmids containing the gfp gene.

GFP production was determined in M. extorquens cells growing in CHOI medium. Cells were initially grown in 50 ml CHOI medium until the mid-exponential phase (OD₆₀₀=0.8). A 2% inoculum was then used to start the growth curve in 200 ml CHOI medium during which samples were taken for measurement of OD₆₀₀, fluorescence and dry weight.

Fluorescence of cell suspensions was determined in a Cytofluor 2300 System (Millipore) under excitation and emission wavelengths of 485 and 530 nm, respectively.

The concentration of GFP was calculated based on a linear relationship between concentration and fluorescence determined for solutions of purified GFP (Quantum Biotechnologies). GFP yield is reported as GFP concentration (μg or mg as indicated) per unit of dry weight (g).

Determination of Cu Concentration in Solution

Cells grown in the presence of Cu were harvested by centrifugation and the supernatant fluid was collected for Cu analysis. Concentrated H₂SO₄ (93%) (0.5 ml) was added to 9.5 ml of supernatant fluid in order to maintain the pH lower than 2.0 (thus preventing Cu precipitation) for Cu ions analysis using inductively coupled plasma-atomic spectrometer (ICP-AS; Thermo Jarel Ash, Trace Scan). The result was corrected with the appropriate dilution factor and referred to as the final soluble Cu concentration. The total Cu concentration added to the medium at preparation was determined likewise by adding 5% (v/v) concentrated H₂SO₄ to the medium and then analyzing using ICP-AS.

Efficiency of Transformation of M. extorquens by Electroporation

An essential step in achieving the expression and stability of heterologous genes in methylotrophic hosts is through the use of suitable broad-host-range vectors. The plasmids used here, pJB3KmD, pRK310 and pVK101, are derived from broad-host-range vectors which were developed for Gram-negative bacteria [Knauf et al. (1982) Plasmid 8:45-54; Ditta et al. (1985) Plasmid 13:1349-153; Blatny et al. (1997) Appl. Environ. Microbiol. 63:370-379]. Table 2 shows the time constant values obtained, as well as the efficiency of transformation for the different constructs used to transform M. extorquens. An overall improvement was observed on the efficiency of transformation of each plasmid in the following order; pJB3KmD<pRK310<pVK101. While the time constant values were within the range obtained by Toyama et al. [Toyama et al. (1998) FEMS Microbiol. Lett. 166(1):1-7] when applying similar electroporation conditions, the efficiency of transformation observed in the present invention for pRK310 (˜10³ cells μg⁻¹ DNA) was at least two orders of magnitude lower than that obtained by them. This value was, however, dose to that obtained by Ueda et al. [Ueda et al. (1991) Ann. N. Y. Acad. Sci. 646:99-105] when they electroporated M. extorquens with pLA2917. Such differences might be due to strain differences or to diverse conditions used for preparing the electro-competent cells, as well as due to specific electroporation conditions such as the time constant produced after each energy discharge. Nonetheless, the transformation efficiencies obtained in the present invention are high enough for practical use in genetic manipulation. The cosmid pVK100 (which resembles pVK101 except for the presence of the cos factor in pVK100) was shown to be mobilized from E. coli strains into M. extorquens AMI (previously known as Pseudomonas sp. AM1) by conjugation at frequencies of 10⁻¹ to 10⁻² [Fulton et al. (1984) J. Bacteriol. 160(2):718-723]; these frequency values were lower than the ones observed in the present invention. There are very few reports in the literature on the use of electroporation as a means of introducing DNA into M. extorquens. Although conjugation has been the preferred technique for transforming methylotrophic bacteria, electroporation was proven here to be a faster and less laborious technique.

TABLE 2
Time constant produced and efficiency of transformation of
M. extorquens by electroporation

| | Time | |
|---|---|---|
| | constant | Efficiency of transformation |
| Plasmid | (ms)′ | (cells pg⁻¹ DNA) |
| pJB3KmD | 8.9 | 1.2 × 10² |
| pRK310 | 8.8 | 2.3 × 10³ |
| pVK101 | 8.6 | 1.1 × 10⁴ |
| PLac-GFP-pJB3KmD | 8.7 | 0.8 × 10² |
| PLac-GPP-pRK310 | 8.8 | 3.6 × 10³ |
| PmmoX-GFP-pRK310 | 8.7 | 0.7 × 10² |
| PmmoX-GFP-pVK101 | 8.1 | 2.0 × 10³ |

′Time of exposure of cells to the high field strength applied.

GFP Production under the Control of the lacZ Promoter

The gfp gene used in the present invention originated from a modified construct of the wild-type GFP [Quantum Biotechnologies, Inc. (1998) Autofluorescent Proteins: Applications Manual. 11NO98]. Its transcription was under the regulation of the lacZ promoter, of the soluble monooxygenase gene duster promoter mmoX [Nielson et al. (1997) Mol. Microbiol. 25(2):399-409] or the methanol dehydrogenase promoter, pmxaF of M. extorquens AM1 (Marx et al., (2001) Microbiology 147, 2065-2075). The lacZ promoter has been successfully used for the expression in GFP by several bacteria [Bermudez et al. (1999) Methods Enzymol. 302:285-295]. The fusion of LacZ regulative elements in constructs containing mosquitocidal endotoxins gene (crylVB) from Bacillus thuringiensis led to a significant increase of crylVB gene expression in the obligate methylotroph Methylobacillus flagellatum [Marchenko et al. (2000) J. Ind. Microbiol. Biotechnol. 24(1);14-18]. However, the absence of the lacl gene gives rise to a constitutive phenotype and thus the lac promoter is induced constitutively even without inducers [Park et al. (1999) J. Microbiol. Biotechnol. 9(6)811-819].

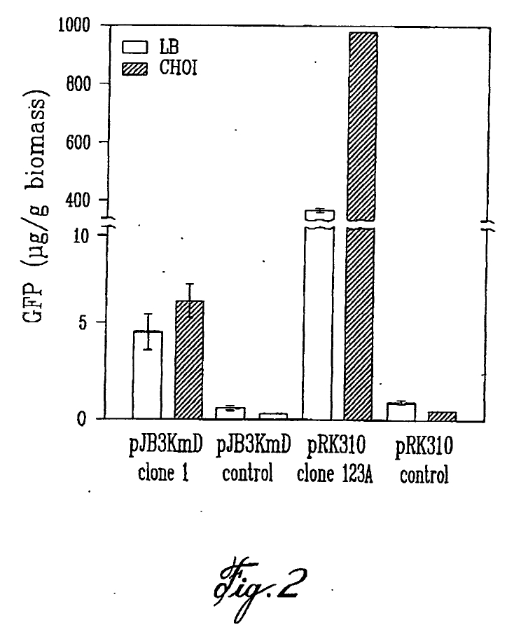

The lac promoter was recognized by M. extorquens in the present invention, in accordance with previous findings [Toyama et al. (1998) FEMS Microbiol. Left. 166(1):1-7]. was also found to be constitutively induced. An interesting observation arose from the comparison between the fluorescence produced by clones of M. extorquens carrying the GFP gene in either pJB3KmD or pRK310 (FIG. 2). The use of the latter led to at least a 100-fold improvement in the amount of GFP produced by each clone.

An important difference was also observed In the amount of GFP produced by clones growing in either LB or CHOI medium (FIG. 2). Independently of the construct used, an improvement of at least 30% in the yield of GFP was obtained when cells grew in CHOI medium. The CHOI medium was described as the ideal medium to obtain high biomass titers of M. extorquens [Bourque et al. (1995) Appl. Microbiol. Biotechnol. 44(3-4):367-376]. The hypothesis that nutritional limitations may interfere with the production of GFP or with its chromophore activity [Tsien (1998) Annu. Rev. Biochem. 67:509-544] should be further investigated in order to explain the significant difference in yields of GFP depending on the medium used.

GFP Production under the Control of mmoX Promoter

In the present invention, M. extorquens was transformed with constructs containing the gfp gene under the control of the mmoX promoter present in the soluble methane monooxygenase (sMMO) operon of another methylotrophic bacterium, Methylosinus trichosporium OB3b. In this microorganism, sMMO catalyzes the oxidation of methane to methanol. The transcriptional regulation of the smmo gene is known to be copper-dependent; sMMO is expressed only under conditions in which the copper-to-biomass ratio is low. This allows for a strict control of the expression of the gene under its promoter by controlling the concentration of Cu in the medium [Nielsen et al. (1997) Mol. Microbiol. 25(2):399-409].

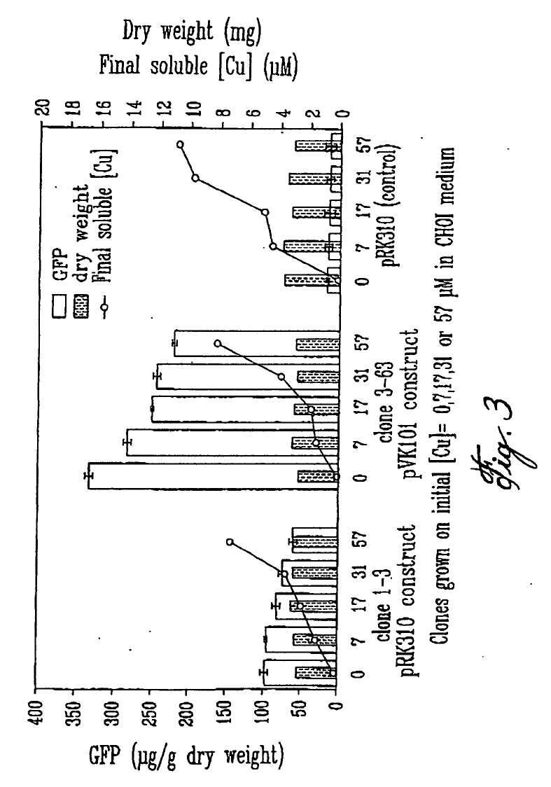

Transformed M. extorquens cells were grown in defined medium (CHOI) so that the effect of varying the concentrations of Cu²⁺ on the production of GFP could be determined (FIG. 3). was found that the promoter was recognized by M. extorquens, therefore, allowing for the expression of GFP. The increase in the initial Cu²⁺ concentration (from 0 to 57 μM) did not interfere with cell growth (as observed by the final dry weight). However, expression of GFP by clones carrying both pmmoX-GFP-pRK310 and pmmoX-GFP-pVK101 was not strongly controlled by the presence of Cu in the medium and repression was observed to some extent when the initial concentation of Cu in the medium was increased up to 57 μM.

Copper specification in the medium and its effect on the activity of sMMO is also relevant to understanding smmo regulation. Morton et al. [Morton et al. (2000) Appl. Environ. Microbiol. 66(4):1730-1733] found no detectable sMMO activity when >2.63 μM Cu g⁻¹ of protein was present. Moreover, different sMMO activities were observed, depending on the type of Cu complex present, which could be explained by the inability of cells to actively transport Cu complexes into the cells, thus reducing Cu bioavailability. In the present invention, analysis of the soluble Cu present in the medium at the end of the cell growth experiments revealed that between 11 and 21% of the Cu added to medium was present in its free form (FIG. 3). The remainder Cu was probably precipitated as oxides, hydroxides or ligand complexes, or accumulated by the cells. While the effect of different species of Cu could affect the regulation of pmmoX, the final concentrations of free Cu in the medium should be high enough to totally inhibit pmmoX expression. However, a reduction of 41 and 33% in GFP production was observed when the final Cu concentration was 2.4 and 3.9 μM Cu mg⁻¹ of biomass (for the clones 1-3 and 3-63, respectively).

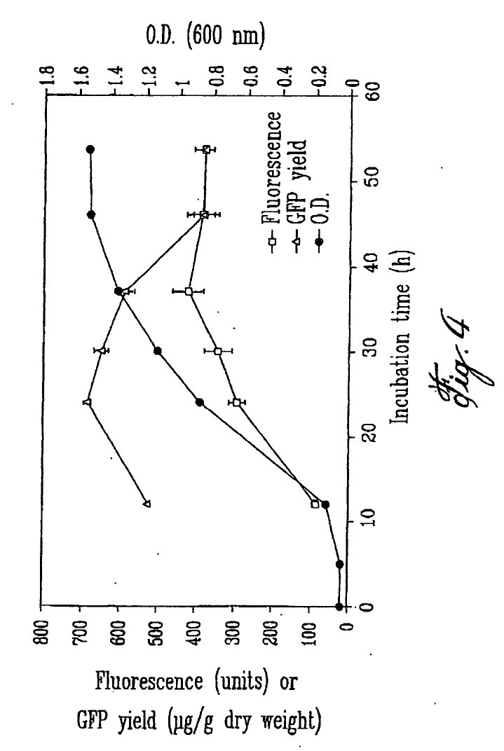

The results for growth of M. extorquens carrying the pmmoX-GFP-pVK101 construct (in the absence of Cu) (FIG. 4) showed that the yield of GFP during growth reached its maximum at mid-exponential phase (about 700 μg of GFP g⁻¹ of biomass) and decreased as the culture reached the early stationary phase of growth (350 μg of GFP g⁻¹ of biomass at stationary phase). Since the fluorescence of cells remained constant during the stationary phase, this suggested that the reduced GFP yield observed may be due to cessation of GFP production during this phase. There could be several factors related to the growth conditions of M. extorquens carrying pmmX-GFP-pVK101 (including O₂ or redox potential limitations, which are known to dramatically affect the maturation of GFP [Tsien (1998) Annu. Rev. Biochem. 67:509-544]) that could explain the apparent end or slowing down of GFP production during stationary phase.

GFP has now been used as a model heterologous protein in order to identify suitable vectors as well as efficient promoters for M. extorquens. pRK310, pVK101 and PCM110 constructs containing Lac, mmoX and mxaF promoters are valuable expression systems for GFP and the expression of other industrially more important, genes in this bacterium should now be more easily accomplished.

GFP Production under the Control of the mxaF Promoter

The strategy used to create pmxaF-GFP-pCM110 (L23) involved digesting the plasmid pQBI63 (Quantum Biotechnologies) which contains gfp gene, with Clal+Xbal. The Xbal/RBS/GFP/Clal band was excised and purified from agarose gel using the QIAEX™ II Agarose Gel Extraction kit (Qiagen Inc.). Plasmid pCM110 [Marx, C. J. and M. E. Lidstrom. (2001) Microbiology 147, 2065-2075] was digested with Clal+Spel and the Spel/pCM110/Clal band was purified as previously. Ligation of both fragments was performed overnight at 16° C. to give pmxaF-GFP-pCM110.

Clones M123A and 28-163 (harbouring the constructs pLac-GFP-pRK310 and pmxaF-GFP-pCM110, respectively) were grown for 24 h in Erlenmeyer flasks containing tetracycline, as described earlier. An aliquot of the culture growth was then used to inoculate flasks containing fresh medium without tetracycline (5% inoculum) for another 24-h period. This procedure was repeated for 9 consecutive batches. A control set of experiments consisted of growing the clone in the same conditions, except that tetracycline was always added to the medium. For each batch, GFP concentration and cell dry weight were measured and specific GFP production was determined as previously described.

Recombinant M. extorquens fed-batch cultures were performed using a 20-L continuously stirred baffled fermenter (Chernap, Volkestwill, Switzerland) equipped with pH and pO₂ electrodes (Ingold), a foam sensor, and a mechanical foam breaker. For agitation, the biorector was equipped with 3 Rushton impellers. The dissolved oxygen level was controlled around 15% of saturation by first, increasing agitation speed from 500 rpm to 1,000 rpm and then, by increasing the airflow supply from 7 L/min to 15 L/min. All fed-batch bioreactor experiments were conducted at pH 7.0 and 30° C. Ammonia solution (30%) was used as both pH regulator and nitrogen source, and was added as needed in all fermentations. Chemical antifoam Ucarferm™ (Union Carbide, Dandury, Conn., U.S.A.) was used as needed to control excessive foaming. Fermenter containing 9 L of modified medium CHOI, and 20 mg ml⁻¹ tetracycline in some experiments (see table 4 below), was inoculated using 1-L culture grown in flasks with or without antibiotic depending on the experiment (see table 4 below).

On-line measurement of the methanol concentration in the culture medium was performed using a silicone membrane probe (Bioengineering Inc.) coupled with a semiconductor gas sensor [Bourque, D., et al., (1995) Appl. Microbiol. Biotechnol. 44(3-4), 367-376]. The methanol concentration was kept around 0.05% (v/v) by using an on-off controller for the first 24-h period, and then a PID controller for the rest of the fermentation. Methanol was added using a variable-speed peristaltic pump. Off-gas measurements were performed for O₂ (Servomex Paramagnetic analyzer) and CO₂ (Servomex Infrared analyzer) concentrations.

Fluorescence of cell suspensions derived from the fermenters, dispensed directly or after dilutions with phosphate buffered saline (pH 7.4) in 96-well black polystyrene plats, was determined in a SPECTRAFluor™ Plus (TECAN Austria Gmbh, Grodlg, Austria) under excitation and emission wavelenghts of 485 and 530 nm, respectively. Cell dry weight for those samples was measured using a Moisture Analyzer MA 30 (Sartorius, Canada).

Off-line measurement of methanol concentrations were determined by gas chromatography (GC) using a Varian gas chromatograph CP-3800 (Varian Analytical Instruments, Walnut Creek, Calif., U.S.A.) equipped with a flame ionisation detector and a DB™-5 capillary column (5% phenyl and 95% methyl silicone, 25 m×0.20 mm×0.33 μm; Alltech, Guelph, Ont., Canada). Other conditions were: column temperature, 70° C. for 2 min, 70-105° C. for 3 min and 105° C. for 0.5 min; injector and detector temperatures, 250° C. and 275° C. respectively; carrier gas He at flow rate of 30 m/min.

The feasibility of using microbial heterologous expression systems in industrial scale depends, among other factors, on how stable the recombinant DNA is within the host cell. For the purpose of simplifying the purification of the recombinant product, it is desirable that cells are able to grow in fermenters in the absence of selective pressure such as antibiotics. This can be achieved by using plasmids that are kept in the cells without modifications in their nucleotide sequence, even if they are grown for several generations in the absence of antibiotics. In order to establish the plasmid stability—as determined by the number of generations—of M. extorquens clones M123A and 28-163 (harbouring the constructs pLacGFP-pRK310 and pmxaF-GFP-pCM110, respectively), repeated sequential batch cultures were carried out in the absence of tetracycilne. A 5% v/v sample of the culture was used as a pre-inoculum in fresh medium and incubated for equivalent amount of time. The process was repeated for 9 consecutive batches. While the production of GFP was kept constant for as long as the cells were grown in tetracycline, relative reductions (based on the specific yields of the first batch culture) of 89% and 99% on GFP production was observed for the clones M123A and 28-163 respectively, after 45 generation times in the absence of antibiotic (Table 3). After 15 generations, corresponding to the number of generations obtained in a 65-h fed-batch culture, the specific GFP production decreased 40% and 35%. respectively. These results indicate that the addition tetracycline is desirable to maintain the maximum recombinant protein production in the conditions applied in the experiments. Depending on the nature and potential intrinsic value of the recombinant product, however, the application of such a system at reduced productivity conditions could still be economically justified, as long as the production process consists of batch fermentations.

TABLE 3
Specific GFP yield (mg protein g cell dry weight (CDW)) by clones
M123A and 23-16 growing in repeated sequential batch cultures, in
presence or absence of tetracycline
Specific GFP production (mg/g) ± standard deviation

| | # of | | | Clone 23-16 | |
|---|---|---|---|---|---|
| Batch | gen- | Clone M123A | | Without | |
| cul- | era- | Without | With | tetra- | With |
| ture | tions | tetracycline | tetracycline | cycline | tetracycline |
| 1 | 5 | 0.49 ± 0.04 | 0.20 ± 0.06 | 48 ± 5 | 40 ± 6 |
| 2 | 10 | 0.16 ± 0.06 | 0.3 ± 0.2 | 33 ± 3 | 35 ± 5 |
| 3 | 15 | 0.18 ± 0.01 | 0.45 ± 0.05 | 17 ± 2 | 32 ± 4 |
| 4 | 20 | 0.12 ± 0.02 | 0.33 ± 0.01 | 19 ± 2 | 36 ± 3 |
| 5 | 25 | 0.12 ± 0.01 | 0.06 ± 0.04 | 12 ± 2 | 33 ± 3 |
| 6 | 30 | 0.2 ± 0.1 | 0.22 ± 0.02 | 3.0 ± 0.5 | 32 ± 2 |
| 7 | 35 | 0.14 ± 0.06 | 0.25 ± 0.03 | 2.7 ± 0.9 | 43 ± 5 |
| 8 | 40 | 0.05 ± 0.01 | 0.349 ± 0.002 | 1.5 ± 0.6 | 38 ± 7 |
| 9 | 45 | 0.054 ± 0.005 | 0.26 ± 0.03 | 0.3 ± 0.2 | 37 ± 9 |

Although shake flask experiments may be useful in determining expression levels of recombinant proteins by recombinant cells (fed-batch cultures), these expression levels may not be comparable to the levels obtained under fed-batch conditions. Changes in growth parameters such as dissolved oxygen, substrate availability and agitation rate can have a profound effect on the expression levels of recombinant proteins [Glick, B. R. (1995) Biotechnol. Adv. 13(2), 247-261]. Therefore, it is necessary to ascertain the effect of scale up of M. extorquens in fed-batch fermentations on the expression levels of recombinant proteins. The following section discusses the performance of M. extorquens as a heterologous system when grown to high cell densities in fed-batch fermenters.

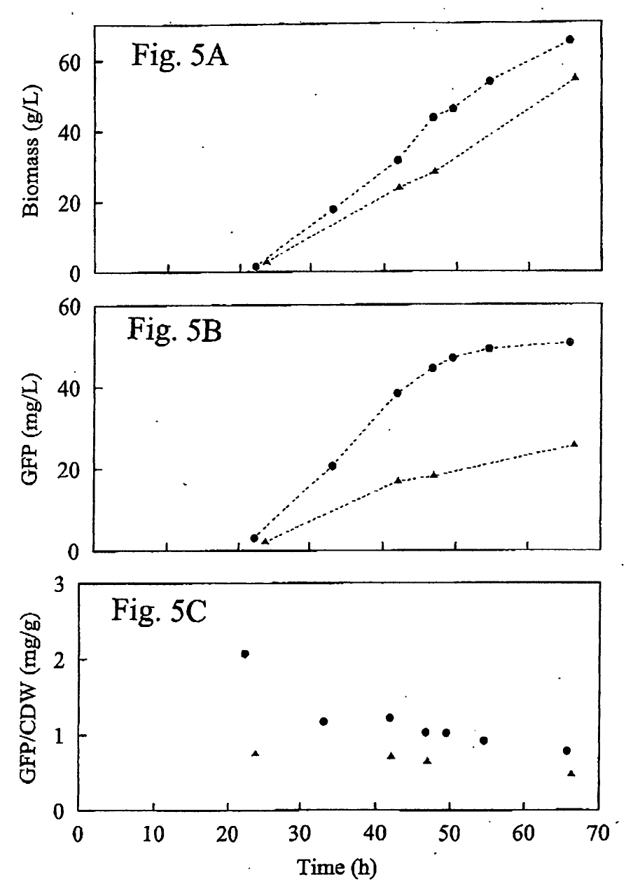

FIG. 5A depicts the growth of M. extorquens done M123A harbouring me pLac-GFP-pRK310 construct in a 20-L fed-batch bioreactor, in the presence or absence of tetracycline. The total biomass production and growth rates (represented by calculated μₘₐₓ, in Table 4) in both conditions were similar. After a 20-h incubation period, cells in the fermenter entered the exponential growth phase, which lasted for at least the next 40 hours. However, the GFP production was markedly reduced when cells were grown in the absence of antibiotics (FIG. 5B), leading to a total specific GFP yield of up to 50% less than that observed for cells growing in medium containing tetracycline (FIG. 5C and Table 4). The reduction levels were comparable to that obtained in flask batches for this clone: after 15 generations, about 40% of the production capacity of the clone is lost by the lack of selective pressure in the medium. These results indicate that there may be one or a combination of factors that affect the recombinant protein synthesis. Among them, the metabolic load, or changes in the metabolism of the host microorganism in ways that may impair the organism's normal metabolic function [Glick, B. R. (1995) Biotechnol. Adv. 13(2), 247-261]. A great amount of energy may be required to maintain the presence of the newly introduced plasmid DNA in a host cell and to produce/overproduce a foreign (recombinant) protein. This may lead to plasmid segregational instability in the absence of selective pressure. When this occurs, the plasmid-less cells out compete the plasmid-bearing cells, resulting in the ultimate decrease in the final yield of recombinant protein [Glick, B. R. (1995) Biotechnol. Adv. 13(2), 247-261; and Zabrsde, D. W. and E. J. Arcud. (1986) Enzyme Microb. Technol. 8, 706-716,4]. Other factors such as oscillations in the carbon source concentration [Lin, H. Y. and P. Neubauer. (2000) J. Biotechnol. 79, 27-37] and oxygen fluctuations [Namdev, P. K., et al., (1993) Biotechnol. Bioeng. 41, 666-670] could also interfere in both plasmid stability and recombinant protein production.

TABLE 4
Final recombinant protein (GFP) and biomass yields for recombinant
clones M123A and 23-16 grown either in presence (+) or
absence (−) of selective pressure in fed-batch fermentations

| | | | | | Yield¹ | |
|---|---|---|---|---|---|---|
| Strain/ | | | Biomass | | | GFP/ |
| Fermen- | Selective | Length | X | μₘₐₓ | GFP/X | Methanol |
| tation | pressure | (h) | (g/L) | (h⁻⁷) | (mg/g) | (mg/g) |
| M123A | | | | | | |
| 1 | + | 73.5 | 72.1 | 0.183 | 0.8 | 0.2 |
| 3 | − | 66 | 54.1 | 0.185 | 0.5 | 0.1 |
| 5 | − | 66 | 66.2 | 0.185 | 0.4 | 0.1 |
| 23-16 | | | | | | |
| 6 | + | 65 | 60.0 | 0.177 | 42.6 | 11.5 |
| 7 | − | 65 | 60.4 | 0.160 | 40.3 | 14.7 |
| 8* | − | 47 | 54.6 | 0.164 | 67.7 | 17.4 |

*fermentation with the addition of enriched air with pure oxygen;
¹production yields are relative to the biomass or total methanol consumed;
²Maximum growth rate (μmax) is calculated based on the OTR (oxygen transfer rate)

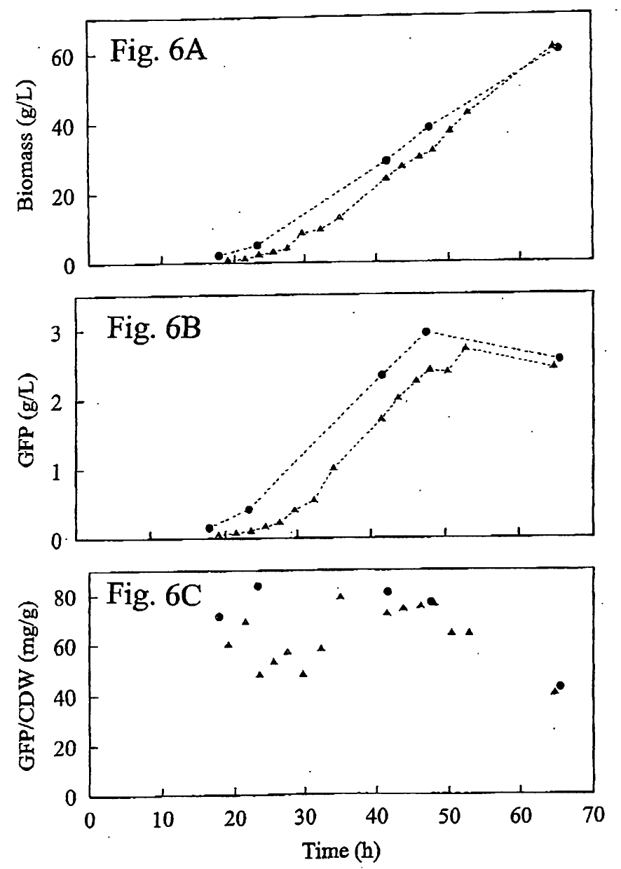

Clones M123A and 23-16 (harbouring pmxaF-GFP-pCM110) possessed similar fed-batch growth characteristics in 20-liter fermenters regardless of the presence or absence of tetracycline (FIGS. 5A and 6A). However, the level of recombinant protein (GFP) production differed significantly. Clone 23-16 produced sixty orders of magnitude more recombinant GFP than clone M123A (FIGS. 5B and 6B; note that y axis units are in mg/L and g/L, respectively), reaching 3 g GFP/L. This higher productivity is related to the presence of a natural promoter to M. extorquens, pmxaF, which controls the expression of the enzyme methanol dehydrogenase in M. extorquens AM1 [Marx, C. J. and M. E. Lidstrom. (2001) Microbiology 147, 2065-2075]. This strong promoter is always “turned on” during the microbial growth due to the constant addition of methanol as the carbon source.

Although growth rates for the clone 23-16 were in general lower than those for the M123A, and slightly lower in experiments run in the absence of antibiotics, GFP production appeared to be unaffected by the presence or absence of selective pressure (FIG. 6B). This suggests that the construct pmxcaF-GFP-pCM110 is indeed more stable than the pLac-GFP-pRK310 construct in M. extorquens cells. The fact is confirmed by the total GFP production yield obtained at the end of the fermentation in the absence of antibiotic (fermentation #7, Table 4), which was very similar to that in the presence of antibiotic (fermentation #6). These results contrast, however, with those obtained in flask experiments where after approximately 15 generations, about 35% of the GFP yield is lost when cells grow in the absence of antibiotics. This difference could be due to very different growth conditions in fed-batch fermentations that do not reflect those of batch systems, emphasizing the need for scale-up optimization. is well known that the number of generations achieved in the fermenter, as well as temperature and pH conditions, agitation, aeration and pressure are factors that affect bioprocesses by scale [Thiry, M. and D. Cingolani. (2002) Trends Biotechnol. 20(3), 103-105].

Mass transfer limitations in the fermenter can result in growth limitations derived from irregular distribution of oxygen to cells. This is particularly true in fed-batch processes, where dissolved oxygen is often a limiting factor if a high growth rate is reached [Thiry, M. and D. Cingolani. (2002) Trends Biotechnol. 20(3), 103-105]. One way of overcoming this problem is by enriching air with pure oxygen, therefore increasing the dissolved oxygen supply without significant increasing the dissolved carbon. Experiment #8 was carried out with this approach as the objective. Indeed, GFP yields were increased considerably (68% higher than when non-enriched air was used, experiment #7), despite the similar maximum growth rate obtained (Table 4). The total amount of biomass obtained was about 10% lower in this condition, but the rate of GFP produced per methanol consumed (17.4 mg GFP/g MeOH) was the highest for all the experiments run, indicating that the metabolism of cells was shifted to producing more GFP for the same amount of MeOH added. This suggests that dissolved oxygen rate in the bioreactor might be an important controlling step in the production of recombinant protein in fed-batch fermentations. The limited amount of dissolved oxygen in the growth medium is often insufficient for both optimal host cell metabolism maintenance and expression. In experiments with E. coli, whenever the dissolved oxygen was rapidly reduced the number of plasmid-containing cells decreased to less than 1% of the cell population, even when the selective pressure was present If oxygen limitations exist even in a transient state throughout the culture, the foreign plasmid may become unstable and lost from the recombinant cells, thereby reducing the yield of the recombinant protein [Glick, B. R. (1995) Biotechnol. Adv. 13(2), 247-261; and White, M. D., et al., (1994) Bacterial, yeast and fungal cultures: the effect of microorganism type and culture characteristics on bioreactor design and operation. In: Bioreactor System Design (Asenjo, J. A. and J. Merchuk Eds.)1-34. Marcel Dekker, N.Y.].

An esterase (estl) gene of Lactobacillus casel was also successfully over-expressed in Methylobacterium extorquens using its homologous methanol dehydrogenase promoter (PmxaF), and the transformed cells produced the enzyme in its active form.

A high-cell density fed-batch fermentation containing methanol a sole source of carbon and energy was carried out under optimal conditions of dissolved oxygen, pH and temperature. Recombinant M. extorquens containing pCEST was grown at pH 6.8 and 30° C. yielding a cell density of 42 g dry cell weight/L after 48 h of growth. Production of recombinant esterase was observed at 20 h of growth and continued to increase until cell harvest (50 h). The total esterase yield obtained (75.2×10³ units/ml) is 980-fold higher than in wild type L. casel, and 2 to 2.5 times higher than in the E. coli. The recombinant enzymes were purified to homogeneity by a single purification step resulting in high yields. No significant differences in physicochemical and catalytic properties were observed between the recombinant enzyme and the native enzymes.

The classic large-scale fermentation process today is a fed-batch process with high final density based on as cheap a substrate as possible. The advantages of this type of bioreactor over the batch system include the prevention of overflow metabolism and minimization of inhibition effects caused by the main carbon source being added in high quantifies at the start of the fermentation [Liden, G. (2002) Bioprocess Biosyst. Eng. 24, 2732791. While oxygen and nutrient transfer limitations can be of concern when high cell densities are achieved in the fermenter, with increasing chances of segregational plasmid instability, this can be partially overcome by changing operational parameters change such as the supply of pure oxygen to the cultures, as shown in the present application.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of producing a recombinant peptide, a recombinant protein or a product from metabolic engineering using a genetically modified methylotrophic bacterium under the control of a regulated promoter from a methylotrophic microorganism of the same or different species as the methylotrophic bacterium, said method comprising the steps of:
a) introducing into said methylotrophic bacterium an expression vector comprising a polynucleotide sequence, coding for a peptide or a protein under the control of a regulated promoter for allowing production of said peptide or protein or for allowing production of a product from metabolic engineering; and
b) growing said genetically modified methylotrophic bacterium in a minimal salts medium lacking organic sugars and containing methanol as a source of carbon and energy for said bacterium for a time sufficient to allow production of said peptide or protein or said product from metabolic engineering.

2. The method of claim 1 further comprising the step of:
c) regulating expression of said polynucleotide sequence by said promoter.

3. The method of claim 1, wherein said regulated promoter is a metal regulated promoter.

4. The method of claim 2, wherein step c) is effected with a metal ion.

5. The method of any one of claims 1, wherein step b) is effected in an oxygen-enriched medium.

6. The method of claim 5 wherein the oxygen-enriched medium is an oxygen-enriched CHOI medium.

7. The method of any one of claims 1, wherein said methylotrophic bacterium is a facultative methylotroph or an obligate methylotroph.

8. The method of any one of claims 1, wherein said methylotrophic bacterium is of the species Methylobacterium.

9. The method of any one of claims 1, wherein said first methylotrophic microorganism is Methylobacterium extorquens ATCC 55366.

10. The method of any one of claims 1, wherein said polynucleotide sequence is a gene coding for green fluorescent protein.

11. The method of any one of claims 1, wherein said polynucleotide sequence is a gene coding for an enzyme.

12. The method of claim 11, wherein said enzyme reacts with a component within or from said culture medium to produce a biomaterial or a product from metabolic engineering.

13. The method of any one of claims 1, wherein said peptide or protein or said product from metabolic engineering reacts with a component within or from said culture medium to produce a biomaterial.

14. The method of any one of claim 1, wherein said vector is capable of reproduction within said bacterium and said vector is stably maintained within said bacterium during growth and replication of said bacterium, in presence of selective pressure.

15. The method of claim 14, wherein said selective pressure is an antibiotic.

16. The method of claim 1, wherein said vector allows for the expression of said polynucleotide sequence within said methylotrophic bacterium.

17. The method of claim 4, wherein said metal ion is Cu²⁺.

18. The method of any one of claims 1, wherein said promoter is the promoter present in the soluble methane monooxygenase (sMMO) operon of Methylosinus trichosporium OB3b.

19. The method of claim 1, wherein said promoter is pmxaF.

20. The method of claim 1, wherein said vector is pmxaF-GFP-pCM110.

21. The method of any one of claims 1, wherein said vector is pmmoX-GFP-pRK310.

22. The method of any one of claims 1, wherein said vector is pmmoX-GFP-pVK101.

23. The method of claim 2, further comprising the step of:
d) controlling the expression of said polynucleotide sequence with a promoter from a gene from an organism other than a methylotrophic bacterium.

24. The method of claim 23, wherein said vector is pLac-GFP-pJB3KmD.

25. The method of claim 23, wherein said vector is pLac-GFP-pRK310.

26. The method of any one of claims 1, wherein the use can be for high-throughput production of a peptide, protein or product from metabolic engineering.

27. The method of any one of claims 1, wherein the use can be for proteomics-based peptide or protein expression.

28. The method of any one of claims 1, wherein the step of growing said genetically modified methylotrophic bacterium is performed within a flask or fermenter.

29. The method of any one of claims 1, wherein said protein is a polypeptide having >10 amino acid residues in length.

30. The method of any one of claims 1, wherein said peptide is ≦10 amino acid residues in length.

31. An expression vector for producing a recombinant peptide, a recombinant protein or a product from metabolic engineering In a methylotrophic bacterium, wherein said expression vector comprises a polynucleotide sequence coding for a peptide or a protein or allowing production of a product from metabolic engineering, under the control of a metal regulated promoter.

32. An expression vector for producing a recombinant peptide, a recombinant protein or a product from metabolic engineering in a methylotrophic bacterium grown in a minimal salts medium lacking organic sugars and containing methanol, wherein said expression vector comprises a polynucleotide sequence coding for a peptide or a protein or allowing production of a product from metabolic engineering, under the control of a methylotrophic bacterium promoter.


SEQUENCE LISTING (4 sequences)

SEQ ID NO: 1 - DNA, 35 bp, Artificial Sequence
  Primer GFP/BamH1.2
gaatcgggatcctcagttgtacagttcatccatgc

SEQ ID NO: 2 - DNA, 38 bp, Artificial Sequence
  Primer RBS/PstI.2
aacaaactgcagaataattttgtttaactttaagaagg

SEQ ID NO: 3 - DNA, 39 bp, Artificial Sequence
  Primer RBS/MluI
cacgacgcgttgaaataattttgtttaactttaagaagg

SEQ ID NO: 4 - DNA, 32 bp, Artificial Sequence
  Primer GFP/XbaI
tgctctagatcagttgtacagttcatccatgc